(19) United States
(12) Patent Application Publication
(10) Pub. No.: US 2020/0315964 A1
(43) Pub. Date: Oct. 8, 2020

(54) NOVEL SEMI-SOLID PREPARATION

(71) Applicant: CorePharmBio Co., Ltd., Gyeonggi-do (KR)

(72) Inventors: Sang Wook KIM, Gyeonggi-do (KR); Prakash KHADKA, Gyeonggi-do (KR); Young Lae KIM, Gyeonggi-do (KR); Jeong Tae KIM, Seoul (KR); Hong Gi YI, Gyonggi-Do (KR)

(21) Appl. No.: 16/303,762

(22) PCT Filed: May 24, 2017
(86) PCT No.: PCT/KR2017/005378

§ 371 Date: Nov. 21, 2018

(30) Foreign Application Priority Data
May 25, 2016 (KR) 10-2016-0064330

(51) Int. Cl. A61K 9/06; A61K 47/36

(52) U.S. Cl. CPC A61K 9/06; A61K 9/0053; A61K 47/36

(57) ABSTRACT

The present invention relates to a novel preparation. The present invention has an advantage of enabling bitterness of the raw material medicine to be masked by only the physical shape thereof. The present invention breaks away from general knowledge, and is a concept having never been conceived of thus far and is a novel preparation, which can be widely used in the pharmaceutical industry in the future.

### FIELD

The present invention relates to a novel formulation which has not yet been known in the pharmaceutical industry field. More specifically, the present invention relates to a novel semi-solid formulation for oral administration, which takes only advantages of liquid and tablet formulations while eliminating the disadvantages of these formulations.

### BACKGROUND

The present invention relates to the field of design of drug products. The development of drugs is based on the investigation of active pharmaceutical ingredients and the design of drug products. As used herein, the expression “design of drug products” is also referred to as formulation.

As the understanding of in vivo pharmacokinetics (absorption, distribution, metabolism and excretion) of active pharmaceutical ingredients becomes important owing to the development of biopharmaceutics, drug products are designed considering even the functionality (such as speed of onset of action, duration of action, or bioavailability) of active pharmaceutical ingredients based on the preformulation results of the active pharmaceutical ingredients, together with control of the physical or physicochemical properties of the active pharmaceutical ingredients. As used herein, the expression “considering functionality” means drug products by examining, for example, the solubility, lipophilicity, pKa, stability in solution, permeability, in vivo stability or pharmacokinetics (PK) of active pharmaceutical ingredients so as to enable the active pharmaceutical ingredients to exhibit their pharmacological effects in an optimized way.

The design that considers functionality is classified as a system having any function, which is used to effectively deliver a drug into the body and is called “drug delivery system (DDS)”. Regarding drug delivery systems, a method of specifying an active pharmaceutical ingredient and designing an optimized system suitable for the specified active pharmaceutical ingredient is not used, but a method is used which comprises developing a specific system and then adopting the developed system when an active pharmaceutical ingredient suitable for the system is present. Thus, in the pharmaceutical field, such systems have been developed without specifying the kind of active pharmaceutical ingredient. For example, Korean Patent Application Publication No. 2006-0115860 is an invention relating to a formulation, but the claims thereof do not specify an active pharmaceutical ingredient. Generally, active pharmaceutical ingredients have different physical and chemical properties, and thus do not appear to exhibit the same in vivo pharmacokinetics. However, active pharmaceutical ingredients can be largely categorized into fast-release, sustained-release and delayed-release pharmaceutical ingredients, and microscopic and minute details can be suitably designed by suitably controlling known additive components and their contents. Thus, developing drug delivery systems without specifying the kind of active pharmaceutical ingredient as described above is significant in the development of the pharmaceutical industry, and when the significance is taken into consideration, patent protection of any drug delivery system should not be limited to an active pharmaceutical ingredient.

The present invention relates to a novel system which is used in the formulation field, similar to the above-described drug delivery systems. The system of the present invention is related to control of the physical or physicochemical form of an active pharmaceutical ingredient. Specifically, the present invention is a system that physically masks the bitter taste of an active pharmaceutical ingredient when active pharmaceutical ingredient is taken orally.

Currently, formulations for oral administration, which are generally frequently used in the pharmaceutical technology field, are solid formulations, including tablets, capsules and the like. Solid formulations have an active ingredient content per unit formulation, which can be accurately controlled, and these solid formulations are advantageous for distribution and storage and also generally show good patient's compliance. However, for infant or geriatric patients who may feel dysphagia, it is required to find substitute dosage forms other than tablets. For old and feeble persons who hardly take solid formulations or patients who feel dysphagia, liquid formulations can be taken into consideration as a substitute dosage form. Liquid formulations do not pose problems such as dysphagia, but the volume thereof may necessarily be larger than that of solid formulations, and special considerations are required during the storage and distribution of these liquid formulations.

The present invention relates to a novel semi-solid formulation for oral administration, which takes only advantages of the above-mentioned liquid and solid formulations while eliminating the disadvantages of these formulations. The Korean Pharmacopoeia describes gel formulations as semi-solid formulations, but these gel formulations are limited to external use, and the semi-solid formulation for oral administration according to the present invention is a novel formulation which has not yet been known in the field to which the present invention pertains. The present invention relates to the first semi-solid formulation introduced in the pharmaceutical industry field, which is suitable for patients who feel dysphagia, and furthermore, has an excellent ability to mask the bitter taste of an active ingredient having a bitter taste.

### SUMMARY

### Technical Problem

Formulations for oral administration typically include solid formulations such as tablets or capsules, and liquid formulations such as suspensions. Among these formulations, solid formulations have advantages in that they enable the dose of an active pharmaceutical ingredient to be accurately fixed, and can maintain their quality for a long period of time due to their strong resistance to deterioration or contamination. However, solid formulations are inconvenient in that they almost cannot be taken without water, even though there is a difference depending on the final volume of drug products. On the contrary, liquid formulations have an advantage in that they can be taken without having to separately prepare water or the like, but have disadvantages in that they do not ensure uniform administration of an active pharmaceutical ingredient, and are susceptible to deterioration or contamination, and particularly, the bitter taste of an active pharmaceutical ingredient can be felt by the tongue.

The present inventor has made extensive efforts to develop a novel semi-solid formulation that takes only advantages of the above-mentioned solid formulations and liquid formulations.

### Technical Solution

To achieve the above object, the present invention provides the following means:

(1) A semi-solid formulation for oral administration, comprising an active pharmaceutical ingredient and at least one thickener and having a PAR value of 50-70 as calculated by the following equation 1:

$$PAR ( θ ) = {tan}^{- 1} ( \frac{3 \cdot V}{π {r}^{3}} ) \qquad Equation 1$$

wherein

r is the radius of the base circle of an imaginary cone, and

V: the volume (1 cm³) of the imaginary cone.

(2) The semi-solid formulation of (1), wherein the thickener is selected from the group consisting of xanthan gum, locust bean gum, guar gum, tragacanth gum, Arabic gum, gellan gum, karaya gum, ghatti gum, tamarind gum, tara gum, acacia gum, agar, chitosan, carrageenan, gelatin, pectin, alginic acid, sodium alginate, propyleneglycol alginate, hypromellose, hydroxyethyl cellulose, hydroxypropyl cellulose, methyl cellulose, hydroxyethyl methyl cellulose, sodium carboxymethyl cellulose, calcium carboxymethyl cellulose, ethyl cellulose, polyethylene glycol, polyvinyl alcohol, povidone, polyethylene oxide, and carbopol.

(3) The semi-solid formulation of (1) or (2), wherein the thickener is selected from the group consisting of locust bean gum, guar gum and xanthan gum.

(4) The semi-solid formulation of any one of (1) to (3), further comprising carrageenan.

(5) A method for preparing a semi-solid formulation for oral administration according to any one of (1) to (4), the method comprising the steps of:

(A) dissolving a thickener in a solvent to form a solution;

(B) dispersing or dissolving an active pharmaceutical ingredient in a solvent to form a solution; and

(C) mixing the solution of step (A) with the solution of step (B) to form the semi-solid formulation for oral administration.

### Advantageous Effect

The semi-solid formulation according to the present invention masks the bitter taste of an active pharmaceutical ingredient, and is easily swallowed when being taken. Furthermore, the formulation of the present invention is easily discharged from a packing container containing the same, and does not leave residue in the packing container after discharge, indicating that it makes possible to take an exact dose of the active pharmaceutical ingredient.

In particular, when the present invention is applied to an active pharmaceutical ingredient in need of fast-release drug delivery, the active pharmaceutical ingredient shows excellent dissolution rate.

### BRIEF DESCRIPTION OF THE DRAWINGS

FIG. 1 is a schematic view of a formulation according to the present invention. In FIG. 1, A1 represents the area of the base of the formulation, and V1 represents the volume of the formulation.

FIG. 2 is a schematic view of an imaginary cone. In FIG. 2, A2 represents the area of the base circle of the imaginary cone; V2 represents the volume of the imaginary cone; r represents the radius of the base circle of the imaginary cone; h represents the height of the imaginary cone; and θ represents the PAR of equation 1. Specifically, FIG. 2 is a schematic view of an imaginary cone having the same base area and same volume as those of the formulation of the present invention.

### DETAILED DESCRIPTION

The present inventor has discovered by chance that when the above-described parameter conditions are satisfied, the bitter taste of an active pharmaceutical ingredient can be advantageously masked by a physical shape, thereby completing the present invention.

In connection with bitter taste masking, several conventional formulation technologies will now be described.

A syrup formulation is a typical liquid formulation for internal use. In the USA, a syrup formulation is defined as a liquid formulation containing an active pharmaceutical ingredient in a concentrated aqueous solution of sugar or the like. Namely, a syrup formulation necessarily contains a sweetener. Sugar such as white sugar functions to offset the bitter taste of an active pharmaceutical ingredient by a sweet taste to thereby increase the ease of taking medication and reduce rejection when the syrup passes through the throat.

A powder formulation is a powdery or fine-particle formulation. The powder formulation has an advantage over a tablet or capsule formulation in that it can be conveniently taken without water by geriatric or pediatric patients, and has an advantage over a liquid formulation in that an active pharmaceutical ingredient has good stability. However, the bitterness of an active pharmaceutical ingredient is recognized as a difficult problem, and attempts have been made to solve this problem by coating or the like.

Rapidly disintegrating tablets or films are classified as solid formulations which are rapidly disintegrated, usually within several seconds, when being placed on the tongue. These formulations are rapidly disintegrated or dissolved by saliva in the mouth within 1 minute (10 seconds in the case of some formulations), after which an active pharmaceutical ingredient in the formulation is absorbed through oral mucosa and gastrointestinal tract mucosa into the systemic vascular system. However, because these formulations are disintegrated or dissolved in the mouth, masking the taste of the active pharmaceutical ingredient is very important in the medication compliance of patients. For the purpose of hiding the taste, in the pharmaceutical field, an aromatic is added, or technologies such as microencapsulation or nanoencapsulation are used.

The formulations as described above adopt, as a bitter taste masking technology, a conception that adds a separate additive or performs coating so that a bitter taste will not be felt in the mouth after which the drug product can move to the gastrointestinal tract.

However, the present invention adopts a method which is completely different from the conventional bitter taste masking conception as described above. According to the present invention, a bitter taste is masked by a physical shape that satisfies novel parameter conditions.

The present invention is characterized in that at least one thickener is added so as to satisfy novel parameter conditions.

The thickener is an additive that is added to a gel formulation. The gel formulation is not listed in the Korean Pharmacopoeia, but may be defined as a semi-solid system comprising a large organic molecule impregnated with a suspension or liquid containing inorganic fine particles. The gel formulation is generally prepared by uniformly dispersing an organic polymer in a dispersion medium such that the boundary between the dispersed phase and the dispersion medium is not distinct, in which the organic polymer functions as a thickener. The thickener may be selected from among synthetic polymers and natural polymers. The natural polymers include, for example, red algae polysaccharides, glucomannan, galactomannan, fermented polysaccharides, brown algae polysaccharides, extracts of marine invertebrate animals, starch, natural fruit extracts, plant fiber derivatives, kelp, natural plant exudates and resinous gums.

In the prior art, the gel formulation has been used mainly as a formulation for skin external use together with a cream or ointment formulation. For example, a product obtained by dispersing a polyethylene resin in a dispersion medium such as water, alcohol or oil was used as an ointment formulation.

However, the present invention breaks from this conventional conception and applies this thickener to a formulation for oral administration.

According to the present invention, at least one of the thickeners as described above is used. Preferably, the formulation according to the present invention comprises at least one selected from among xanthan gum, locust bean gum, guar gum, tragacanth gum, Arabic gum, gellan gum, karaya gum, ghatti gum, tamarind gum, tara gum, Acacia gum, agar, chitosan, carrageenan, gelatin, pectin, alginic acid, sodium alginate, propyleneglycol alginate, Hypromellose, hydroxyethyl cellulose, hydroxypropyl cellulose, methyl cellulose, hydroxyethyl methyl cellulose, sodium carboxymethyl cellulose, calcium carboxymethyl cellulose, ethyl cellulose, polyethylene glycol, polyvinyl alcohol, povidone, polyethylene oxide, and carbopol. However, the selected thickener should not be used in a random manner, and the component and content thereof should be determined so as to satisfy a PAR value of 50-70 as calculated by the following equation 1:

$$PAR ( θ ) = {tan}^{- 1} ( \frac{3 \cdot V}{π {r}^{3}} ) \qquad Equation 1$$

wherein r is the radius of the base circle of an imaginary cone, and

V: the volume (1 cm³) of the imaginary cone.

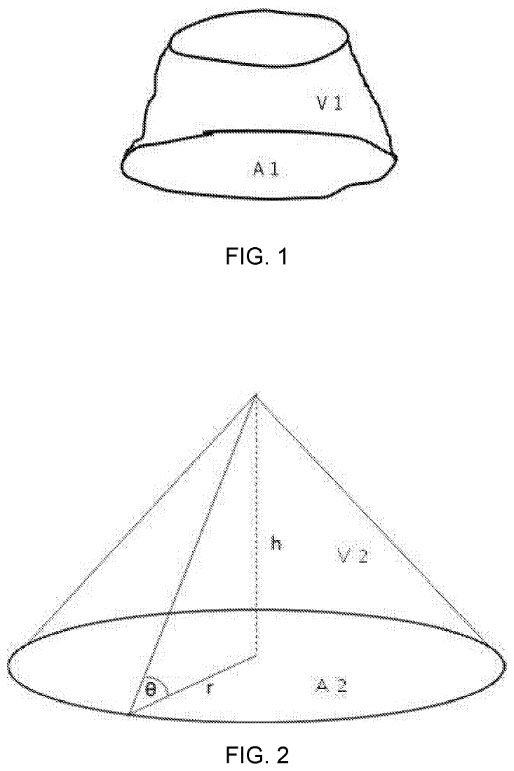

PAR (Pseudo-Angle of Repose) is a value used to express the spreadability of the formulation of the present invention as data, and is an imaginary value obtained by placing the formulation having a certain volume on a flat base, measuring the contact area (A1) between the formulation and the base, drawing an imaginary circle having the same area as the contact area, drawing an imaginary circle based on the area (A2) of the circle and the volume (V1) of the formulation, and measuring the angle between the base and the hypotenuse of the cone (see FIGS. 1 and 2).

The symbols used in FIGS. 1 and 2 are defined as follows.

r (the radius of the base circle of an imaginary cone); h (the height of the imaginary cone); θ (the pseudo-angle of repose (PAR));

A1 (the area of the base of the formulation)=A2 (the area of the base circle of the imaginary cone);

V1 (the volume of the formulation)=V2 (the volume of the imaginary cone)=V(1 cm³).

As the PAR value increases, the contact area (A1) between the formulation and the base decreases. This suggests that the formulation has high strength so that it can readily maintain its shape. Thus, it can be determined that the larger the PAR value, the higher is the strength of the formulation.

Although an exact mechanism is not understood, the present inventor has surprisingly discovered that when the PAR value is 50-70, various advantages can all be satisfied. Specifically, in this case, the bitter taste of an active pharmaceutical ingredient when taking the active pharmaceutical ingredient is significantly reduced, swallowing of the formulation comprising the active pharmaceutical ingredient is good, the formulation is easily discharged from a packing container, and the formulation does not remain in the packing container after discharge. The present invention takes only advantages of liquid and solid formulations while eliminating the disadvantages of these formulations. For this purpose, it is required to take advantages of a liquid formulation that can be easily swallowed by a patient who feels dysphagia, and also advantages of a tablet formulation that has a small unit formulation size and is advantageous for storage and distribution. However, the present inventor has found that all the above-described advantages are not achieved when a viscous formulation is merely prepared using a conventional polymer, and that only when the PAR is satisfied, the advantages of the two formulations can be achieved, thereby completing the present invention. The present invention relates to a novel formulation that has not yet been known in the prior art, and an active ingredient (active pharmaceutical ingredient), which may be contained in the novel formulation, is not limited. Particularly, when the present invention is applied to an active ingredient having a bitter taste, it achieves an additional excellent effect of masking the bitter taste. Furthermore, the present invention may be applied not only to a fast-release drug delivery system, but also to a sustained-release or delayed-release drug delivery system, by suitably selecting additives.

According to the present invention, the formulation may further comprise, in addition to the thickener, other additives, including a dispersion medium and a preservative.

The dispersion medium may be suitably selected depending on the physical and chemical properties of the active pharmaceutical ingredient and the thickener, and may be one selected from among known dispersion media, including water, alcohol and oil. For example, when a water-soluble natural thickener is selected, purified water may be selected as the dispersion medium.

The preservative that is used in the present invention may be one or more known preservatives. For example, the preservative may be methyl p-hydroxybenzoate or propyl p-hydroxybenzoate.

The contents of additives that are used in the present invention can be suitably determined by those skilled in the art. Namely, with reference to the following examples and test examples, those skilled in the art can determine a specific composition satisfying the PAR defined in the present invention. Thus, it should be understood that the present invention is not limited to a composition containing specific components.

### EXAMPLES

Hereinafter, the present invention will be illustrated by the following non-limiting examples.

### Preparation Examples 1 to 30

Povidone was dissolved in purified water (1), and then acetaminophen was added and dispersed therein to prepare a dispersion of the active ingredient. In addition, a mixture of at least one thickener, xylitol, stevioside and a preservative was added to purified water (2), and then stirred. After the added components were completely dissolved, the above-prepared active ingredient dispersion was added to the stirred solution, and then uniformly dispersed using a homogenizer for 5 minutes. Finally, bubbles were removed from the dispersion.

Specific compositions of Preparation Examples 1 to 30 are shown in Tables 1 to 6 below.

TABLE 1
Compositions [weight ratio (w/w %)] of Preparation Examples 1 to 5

| | Preparation | Preparation | Preparation | Preparation | Preparation |
|---|---|---|---|---|---|
| Components | Example 1 | Example 2 | Example 3 | Example 4 | Example 5 |
| Acetaminophen | 3.200 | 3.200 | 3.200 | 3.200 | 3.200 |
| Povidone (PVP-12) | 2.000 | 2.000 | 2.000 | 2.000 | 2.000 |
| Purified water (1) | 20.000 | 20.000 | 20.000 | 20.000 | 20.000 |
| Carrageenan | 0.000 | 0.000 | 0.000 | 0.000 | 0.000 |
| Locust bean gum | 0.067 | 0.133 | 0.200 | 0.267 | 0.333 |
| Xanthan gum | 0.067 | 0.133 | 0.200 | 0.267 | 0.333 |
| Xylitol | 28.023 | 28.023 | 28.023 | 28.023 | 28.023 |
| Stevioside | 0.100 | 0.100 | 0.100 | 0.100 | 0.100 |
| Methyl p- | 0.005 | 0.005 | 0.005 | 0.005 | 0.005 |
| hydroxybenzoate | | | | | |
| Propyl p- | 0.005 | 0.005 | 0.005 | 0.005 | 0.005 |
| hydroxybenzoate | | | | | |
| Purified water (2) | 46.533 | 46.400 | 46.267 | 46.133 | 46.000 |
| Sum | 100.00 | 100.00 | 100.00 | 100.00 | 100.00 |

TABLE 2
Compositions [weight ratio (w/w %)] of Preparation Examples 6 to 10

| | Preparation | Preparation | Preparation | Preparation | Preparation |
|---|---|---|---|---|---|
| Components | Example 6 | Example 7 | Example 8 | Example 9 | Example 10 |
| Acetaminophen | 3.200 | 3.200 | 3.200 | 3.200 | 3.200 |
| Povidone (PVP-12) | 2.000 | 2.000 | 2.000 | 2.000 | 2.000 |
| Purified water (1) | 20.000 | 20.000 | 20.000 | 20.000 | 20.000 |
| Carrageenan | 0.000 | 0.033 | 0.033 | 0.033 | 0.033 |
| Locust bean gum | 0.400 | 0.067 | 0.133 | 0.200 | 0.267 |
| Xanthan gum | 0.400 | 0.067 | 0.133 | 0.200 | 0.267 |
| Xylitol | 28.023 | 28.023 | 28.023 | 28.023 | 28.023 |
| Stevioside | 0.100 | 0.100 | 0.100 | 0.100 | 0.100 |
| Methyl p- | 0.005 | 0.005 | 0.005 | 0.005 | 0.005 |
| hydroxybenzoate | | | | | |
| Propyl p- | 0.005 | 0.005 | 0.005 | 0.005 | 0.005 |
| hydroxybenzoate | | | | | |
| Purified water (2) | 45.867 | 46.500 | 46.367 | 46.233 | 46.100 |
| Sum | 100.00 | 100.00 | 100.00 | 100.00 | 100.00 |

TABLE 3
Compositions [weight ratio (w/w %)] of Preparation Examples 11 to 15

| | Preparation | Preparation | Preparation | Preparation | Preparation |
|---|---|---|---|---|---|
| Components | Example 11 | Example 12 | Example 13 | Example 14 | Example 15 |
| Acetaminophen | 3.200 | 3.200 | 3.200 | 3.200 | 3.200 |
| Povidone (PVP-12) | 2.000 | 2.000 | 2.000 | 2.000 | 2.000 |
| Purified water (1) | 20.000 | 20.000 | 20.000 | 20.000 | 20.000 |
| Carrageenan | 0.033 | 0.033 | 0.067 | 0.067 | 0.067 |
| Locust bean gum | 0.333 | 0.400 | 0.067 | 0.133 | 0.200 |
| Xanthan gum | 0.333 | 0.400 | 0.067 | 0.133 | 0.200 |
| Xylitol | 28.023 | 28.023 | 28.023 | 28.023 | 28.023 |
| Stevioside | 0.100 | 0.100 | 0.100 | 0.100 | 0.100 |
| Methyl p- | 0.005 | 0.005 | 0.005 | 0.005 | 0.005 |
| hydroxybenzoate | | | | | |
| Propyl p- | 0.005 | 0.005 | 0.005 | 0.005 | 0.005 |
| hydroxybenzoate | | | | | |
| Purified water (2) | 45.967 | 45.833 | 46.467 | 46.333 | 46.200 |
| Sum | 100.00 | 100.00 | 100.00 | 100.00 | 100.00 |

TABLE 4
Compositions [weight ratio (w/w %)] of Preparation Examples 16 to 20

| | Preparation | Preparation | Preparation | Preparation | Preparation |
|---|---|---|---|---|---|
| Components | Example 16 | Example 17 | Example 18 | Example 19 | Example 20 |
| Acetaminophen | 3.200 | 3.200 | 3.200 | 3.200 | 3.200 |
| Povidone (PVP-12) | 2.000 | 2.000 | 2.000 | 2.000 | 2.000 |
| Purified water (1) | 20.000 | 20.000 | 20.000 | 20.000 | 20.000 |
| Carrageenan | 0.067 | 0.067 | 0.067 | 0.133 | 0.133 |
| Locust bean gum | 0.267 | 0.333 | 0.400 | 0.067 | 0.133 |
| Xanthan gum | 0.267 | 0.333 | 0.400 | 0.067 | 0.133 |
| Xylitol | 28.023 | 28.023 | 28.023 | 28.023 | 28.023 |
| Stevioside | 0.100 | 0.100 | 0.100 | 0.100 | 0.100 |
| Methyl p- | 0.005 | 0.005 | 0.005 | 0.005 | 0.005 |
| hydroxybenzoate | | | | | |
| Propyl p- | 0.005 | 0.005 | 0.005 | 0.005 | 0.005 |
| hydroxybenzoate | | | | | |
| Purified water (2) | 46.067 | 45.933 | 45.800 | 46.400 | 46.267 |
| Sum | 100.00 | 100.00 | 100.00 | 100.00 | 100.00 |

TABLE 5
Compositions [weight ratio (w/w %)] of Preparation Examples 21 to 25

| | Preparation | Preparation | Preparation | Preparation | Preparation |
|---|---|---|---|---|---|
| Components | Example 21 | Example 22 | Example 23 | Example 24 | Example 25 |
| Acetaminophen | 3.200 | 3.200 | 3.200 | 3.200 | 3.200 |
| Povidone (PVP-12) | 2.000 | 2.000 | 2.000 | 2.000 | 2.000 |
| Purified water (1) | 20.000 | 20.000 | 20.000 | 20.000 | 20.000 |
| Carrageenan | 0.133 | 0.133 | 0.133 | 0.133 | 0.013 |
| Locust bean gum | 0.200 | 0.267 | 0.333 | 0.400 | 0.067 |
| Xanthan gum | 0.200 | 0.267 | 0.333 | 0.400 | 0.067 |
| Xylitol | 28.023 | 28.023 | 28.023 | 28.023 | 28.023 |
| Stevioside | 0.100 | 0.100 | 0.100 | 0.100 | 0.100 |
| Methyl p- | 0.005 | 0.005 | 0.005 | 0.005 | 0.005 |
| hydroxybenzoate | | | | | |
| Propyl p- | 0.005 | 0.005 | 0.005 | 0.005 | 0.005 |
| hydroxybenzoate | | | | | |
| Purified water (2) | 46.133 | 46.000 | 45.867 | 45.733 | 45.520 |
| Sum | 100.00 | 100.00 | 100.00 | 100.00 | 100.00 |

TABLE 6
Compositions [weight ratio (w/w %)] of Preparation Examples 26 to 30

| | Preparation | Preparation | Preparation | Preparation | Preparation |
|---|---|---|---|---|---|
| Components | Example 26 | Example 27 | Example 28 | Example 29 | Example 30 |
| Acetaminophen | 3.200 | 3.200 | 3.200 | 3.200 | 3.200 |
| Povidone (PVP- | 2.000 | 2.000 | 2.000 | 2.000 | 2.000 |
| 12) | | | | | |
| Purified water (1) | 20.000 | 20.000 | 20.000 | 20.000 | 20.000 |
| Carrageenan | 0.013 | 0.013 | 0.013 | 0.013 | 0.013 |
| Locust bean gum | 0.133 | 0.200 | 0.267 | 0.333 | 0.400 |
| Xanthan gum | 0.133 | 0.200 | 0.267 | 0.333 | 0.400 |
| Xylitol | 28.023 | 28.023 | 28.023 | 28.023 | 28.023 |
| Stevioside | 0.100 | 0.100 | 0.100 | 0.100 | 0.100 |
| Methyl p- | 0.005 | 0.005 | 0.005 | 0.005 | 0.005 |
| hydroxybenzoate | | | | | |
| Propyl p- | 0.005 | 0.005 | 0.005 | 0.005 | 0.005 |
| hydroxybenzoate | | | | | |
| Purified water (2) | 46.387 | 46.253 | 46.120 | 45.987 | 45.853 |
| Sum | 100.00 | 100.00 | 100.00 | 100.00 | 100.00 |

### Preparation Examples 31 to 60

Compositions of Preparation Examples 31 to 60 were prepared in the same manner as Preparation Examples 1 to 30, except that cetirizine hydrochloride was used as an active ingredient and that meglumine was used in place of povidone. Finally, bubbles were removed.

Specific compositions of Preparation Examples 31 to 60 are shown in Tables 7 to 12.

TABLE 7
Compositions [weight ratio (w/w %)] of Preparation Examples 31 to 35

| | Preparation | Preparation | Preparation | Preparation | Preparation |
|---|---|---|---|---|---|
| Components | Example 31 | Example 32 | Example 33 | Example 34 | Example 35 |
| Cetirizine | 0.400 | 0.400 | 0.400 | 0.400 | 0.400 |
| hydrochloride | | | | | |
| Meglumine | 2.000 | 2.000 | 2.000 | 2.000 | 2.000 |
| Purified water (1) | 20.000 | 20.000 | 20.000 | 20.000 | 20.000 |
| Carrageenan | 0.000 | 0.000 | 0.000 | 0.000 | 0.000 |
| Locust bean gum | 0.067 | 0.133 | 0.200 | 0.267 | 0.333 |
| Xanthan gum | 0.067 | 0.133 | 0.200 | 0.267 | 0.333 |
| Xylitol | 30.823 | 30.823 | 30.823 | 30.823 | 30.823 |
| Stevioside | 0.100 | 0.100 | 0.100 | 0.100 | 0.100 |
| Methyl p- | 0.005 | 0.005 | 0.005 | 0.005 | 0.005 |
| hydroxybenzoate | | | | | |
| Propyl p- | 0.005 | 0.005 | 0.005 | 0.005 | 0.005 |
| hydroxybenzoate | | | | | |
| Purified water (2) | 46.533 | 46.400 | 46.267 | 46.133 | 46.000 |
| Sum | 100.00 | 100.00 | 100.00 | 100.00 | 100.00 |

TABLE 8
Compositions [weight ratio (w/w %)] of Preparation Examples 36 to 40

| | Preparation | Preparation | Preparation | Preparation | Preparation |
|---|---|---|---|---|---|
| Components | Example 36 | Example 37 | Example 38 | Example 39 | Example 40 |
| Cetirizine | 0.400 | 0.400 | 0.400 | 0.400 | 0.400 |
| hydrochloride | | | | | |
| Meglumine | 2.000 | 2.000 | 2.000 | 2.000 | 2.000 |
| Purified water (1) | 20.000 | 20.000 | 20.000 | 20.000 | 20.000 |
| Carrageenan | 0.000 | 0.033 | 0.033 | 0.033 | 0.033 |
| Locust bean gum | 0.400 | 0.67 | 0.133 | 0.200 | 0.267 |
| Xanthan gum | 0.400 | 0.067 | 0.133 | 0.200 | 0.267 |
| Xylitol | 30.823 | 30.823 | 30.823 | 30.823 | 30.823 |
| Stevioside | 0.100 | 0.100 | 0.100 | 0.100 | 0.100 |
| Methyl p- | 0.005 | 0.005 | 0.005 | 0.005 | 0.005 |
| hydroxybenzoate | | | | | |
| Propyl p- | 0.005 | 0.005 | 0.005 | 0.005 | 0.005 |
| hydroxybenzoate | | | | | |
| Purified water (2) | 45.86667 | 46.5 | 46.36667 | 46.23333 | 46.1 |
| Sum | 100.00 | 100.00 | 100.00 | 100.00 | 100.00 |

TABLE 9
Compositions [weight ratio (w/w %)] of Preparation Examples 41 to 45

| | Preparation | Preparation | Preparation | Preparation | Preparation |
|---|---|---|---|---|---|
| Components | Example 41 | Example 42 | Example 43 | Example 44 | Example 45 |
| Cetirizine | 0.400 | 0.400 | 0.400 | 0.400 | 0.400 |
| hydrochloride | | | | | |
| Meglumine | 2.000 | 2.000 | 2.000 | 2.000 | 2.000 |
| Purified water (1) | 20.000 | 20.000 | 20.000 | 20.000 | 20.000 |
| Carrageenan | 0.033 | 0.033 | 0.067 | 0.067 | 0.067 |
| Locust bean gum | 0.333 | 0.400 | 0.067 | 0.133 | 0.200 |
| Xanthan gum | 0.333 | 0.400 | 0.067 | 0.133 | 0.200 |
| Xylitol | 30.823 | 30.823 | 30.823 | 30.823 | 30.823 |
| Stevioside | 0.100 | 0.100 | 0.100 | 0.100 | 0.100 |
| Methyl p- | 0.005 | 0.005 | 0.005 | 0.005 | 0.005 |
| hydroxybenzoate | | | | | |
| Propyl p- | 0.005 | 0.005 | 0.005 | 0.005 | 0.005 |
| hydroxybenzoate | | | | | |
| Purified water (2) | 45.967 | 45.833 | 46.467 | 46.333 | 46.200 |
| Sum | 100.00 | 100.00 | 100.00 | 100.00 | 100.00 |

TABLE 10
Compositions [weight ratio (w/w %)] of Preparation Examples 46 to 50

| | Preparation | Preparation | Preparation | Preparation | Preparation |
|---|---|---|---|---|---|
| Components | Example 46 | Example 47 | Example 48 | Example 49 | Example 50 |
| Cetirizine | 0.400 | 0.400 | 0.400 | 0.400 | 0.400 |
| hydrochloride | | | | | |
| Meglumine | 2.000 | 2.000 | 2.000 | 2.000 | 2.000 |
| Purified water (1) | 20.000 | 20.000 | 20.000 | 20.000 | 20.000 |
| Carrageenan | 0.067 | 0.067 | 0.067 | 0.133 | 0.133 |
| Locust bean gum | 0.267 | 0.333 | 0.400 | 0.067 | 0.133 |
| Xanthan gum | 0.267 | 0.333 | 0.400 | 0.067 | 0.133 |
| Xylitol | 30.823 | 30.823 | 30.823 | 30.823 | 30.823 |
| Stevioside | 0.100 | 0.100 | 0.100 | 0.100 | 0.100 |
| Methyl p- | 0.005 | 0.005 | 0.005 | 0.005 | 0.005 |
| hydroxybenzoate | | | | | |
| Propyl p- | 0.005 | 0.005 | 0.005 | 0.005 | 0.005 |
| hydroxybenzoate | | | | | |
| Purified water (2) | 46.067 | 45.933 | 45.800 | 46.400 | 46.267 |
| Sum | 100.00 | 100.00 | 100.00 | 100.00 | 100.00 |

TABLE 11
Compositions [weight ratio (w/w %)] of Preparation Examples 51 to 55

| | Preparation | Preparation | Preparation | Preparation | Preparation |
|---|---|---|---|---|---|
| Components | Example 51 | Example 52 | Example 53 | Example 54 | Example 55 |
| Cetirizine | 0.400 | 0.400 | 0.400 | 0.400 | 0.400 |
| hydrochloride | | | | | |
| Meglumine | 2.000 | 2.000 | 2.000 | 2.000 | 2.000 |
| Purified water (1) | 20.000 | 20.000 | 20.000 | 20.000 | 20.000 |
| Carrageenan | 0.133 | 0.133 | 0.133 | 0.133 | 0.013 |
| Locust bean gum | 0.200 | 0.267 | 0.333 | 0.400 | 0.067 |
| Xanthan gum | 0.200 | 0.267 | 0.333 | 0.400 | 0.067 |
| Xylitol | 30.823 | 30.823 | 30.823 | 30.823 | 30.823 |
| Stevioside | 0.100 | 0.100 | 0.100 | 0.100 | 0.100 |
| Methyl p- | 0.005 | 0.005 | 0.005 | 0.005 | 0.005 |
| hydroxybenzoate | | | | | |
| Propyl p- | 0.005 | 0.005 | 0.005 | 0.005 | 0.005 |
| hydroxybenzoate | | | | | |
| Purified water (2) | 46.133 | 46.000 | 45.867 | 45.733 | 46.520 |
| Sum | 100.00 | 100.00 | 100.00 | 100.00 | 100.00 |

TABLE 12
Compositions [weight ratio (w/w %)] of Preparation Examples 56 to 60

| | Preparation | Preparation | Preparation | Preparation | Preparation |
|---|---|---|---|---|---|
| Components | Example 56 | Example 57 | Example 58 | Example 59 | Example 60 |
| Cetirizine | 0.400 | 0.400 | 0.400 | 0.400 | 0.400 |
| hydrochloride | | | | | |
| Meglumine | 2.000 | 2.000 | 2.000 | 2.000 | 2.000 |
| Purified water (1) | 20.000 | 20.000 | 20.000 | 20.000 | 20.000 |
| Carrageenan | 0.013 | 0.013 | 0.013 | 0.013 | 0.013 |
| Locust bean gum | 0.133 | 0.200 | 0.267 | 0.333 | 0.400 |
| Xanthan gum | 0.133 | 0.200 | 0.267 | 0.333 | 0.400 |
| Xylitol | 30.82333 | 30.82333 | 30.82333 | 30.82333 | 30.82333 |
| Stevioside | 0.100 | 0.100 | 0.100 | 0.100 | 0.100 |
| Methyl p- | 0.005 | 0.005 | 0.005 | 0.005 | 0.005 |
| hydroxybenzoate | | | | | |
| Propyl p- | 0.005 | 0.005 | 0.005 | 0.005 | 0.005 |
| hydroxybenzoate | | | | | |
| Purified water (2) | 46.38667 | 46.25333 | 46.12 | 45.98667 | 45.85333 |
| Sum | 100.00 | 100.00 | 100.00 | 100.00 | 100.00 |

### Preparation Examples 61 to 90

Compositions of Preparation Examples 61 to 90 were prepared in the same manner as Preparation Examples 1 to 30, except that Loxoprofen sodium was used as an active ingredient.

Specific compositions of Preparation Examples 61 to 90 are shown in Tables 13 to 18.

TABLE 13
Compositions [weight ratio (w/w %)] of Preparation Examples 61 to 65

| | Preparation | Preparation | Preparation | Preparation | Preparation |
|---|---|---|---|---|---|
| Components | Example 61 | Example 62 | Example 63 | Example 64 | Example 65 |
| Loxoprofen sodium | 3.200 | 3.200 | 3.200 | 3.200 | 3.200 |
| Purified water (1) | 20.000 | 20.000 | 20.000 | 20.000 | 20.000 |
| Carrageenan | 0.000 | 0.000 | 0.000 | 0.000 | 0.000 |
| Locust bean gum | 0.067 | 0.133 | 0.200 | 0.267 | 0.333 |
| Xanthan gum | 0.067 | 0.133 | 0.200 | 0.267 | 0.333 |
| Xylitol | 30.023 | 30.023 | 30.023 | 30.023 | 30.023 |
| Stevioside | 0.100 | 0.100 | 0.100 | 0.100 | 0.100 |
| Methyl p- | 0.005 | 0.005 | 0.005 | 0.005 | 0.005 |
| hydroxybenzoate | | | | | |
| Propyl p- | 0.005 | 0.005 | 0.005 | 0.005 | 0.005 |
| hydroxybenzoate | | | | | |
| Purified water (2) | 46.533 | 46.400 | 46.267 | 46.133 | 46.000 |
| Sum | 100.000 | 100.000 | 100.000 | 100.000 | 100.000 |

TABLE 14
Compositions [weight ratio (w/w %)] of Preparation Examples 66 to 70

| | Preparation | Preparation | Preparation | Preparation | Preparation |
|---|---|---|---|---|---|
| Components | Example 66 | Example 67 | Example 68 | Example 69 | Example 70 |
| Loxoprofen sodium | 3.2 | 3.2 | 3.2 | 3.2 | 3.2 |
| Purified water (1) | 20.000 | 20.000 | 20.000 | 20.000 | 20.000 |
| Carrageenan | 0.000 | 0.033 | 0.033 | 0.033 | 0.033 |
| Locust bean gum | 0.400 | 0.067 | 0.133 | 0.200 | 0.267 |
| Xanthan gum | 0.400 | 0.067 | 0.133 | 0.200 | 0.267 |
| Xylitol | 30.023 | 30.023 | 30.023 | 30.023 | 30.023 |
| Stevioside | 0.100 | 0.100 | 0.100 | 0.100 | 0.100 |
| Methyl p- | 0.005 | 0.005 | 0.005 | 0.005 | 0.005 |
| hydroxybenzoate | | | | | |
| Propyl p- | 0.005 | 0.005 | 0.005 | 0.005 | 0.005 |
| hydroxybenzoate | | | | | |
| Purified water (2) | 45.867 | 46.500 | 46.367 | 46.233 | 46.100 |
| Sum | 100.000 | 100.000 | 100.000 | 100.000 | 100.000 |

TABLE 15
Compositions [weight ratio (w/w %)] of Preparation Examples 71 to 75

| | Preparation | Preparation | Preparation | Preparation | Preparation |
|---|---|---|---|---|---|
| Components | Example 71 | Example 72 | Example 73 | Example 74 | Example 75 |
| Loxoprofen sodium | 3.200 | 3.200 | 3.200 | 3.200 | 3.200 |
| Purified water (1) | 20.000 | 20.000 | 20.000 | 20.000 | 20.000 |
| Carrageenan | 0.033 | 0.033 | 0.067 | 0.067 | 0.067 |
| Locust bean gum | 0.333 | 0.400 | 0.067 | 0.133 | 0.200 |
| Xanthan gum | 0.333 | 0.400 | 0.067 | 0.133 | 0.200 |
| Xylitol | 30.023 | 30.023 | 30.023 | 30.023 | 30.023 |
| Stevioside | 0.100 | 0.100 | 0.100 | 0.100 | 0.100 |
| Methyl p- | 0.005 | 0.005 | 0.005 | 0.005 | 0.005 |
| hydroxybenzoate | | | | | |
| Propyl p- | 0.005 | 0.005 | 0.005 | 0.005 | 0.005 |
| hydroxybenzoate | | | | | |
| Purified water (2) | 45.967 | 45.833 | 46.467 | 46.333 | 46.200 |
| Sum | 100.000 | 100.000 | 100.000 | 100.000 | 100.000 |

TABLE 16
Compositions [weight ratio (w/w %)] of Preparation Examples 76 to 80

| | Preparation | Preparation | Preparation | Preparation | Preparation |
|---|---|---|---|---|---|
| Components | Example 76 | Example 77 | Example 78 | Example 79 | Example 80 |
| Loxoprofen sodium | 3.200 | 3.200 | 3.200 | 3.200 | 3.200 |
| Purified water (1) | 20.000 | 20.000 | 20.000 | 20.000 | 20.000 |
| Carrageenan | 0.067 | 0.067 | 0.067 | 0.133 | 0.133 |
| Locust bean gum | 0.267 | 0.333 | 0.400 | 0.067 | 0.133 |
| Xanthan gum | 0.267 | 0.333 | 0.400 | 0.067 | 0.133 |
| Xylitol | 30.023 | 30.023 | 30.023 | 30.023 | 30.023 |
| Stevioside | 0.100 | 0.100 | 0.100 | 0.100 | 0.100 |
| Methyl p- | 0.005 | 0.005 | 0.005 | 0.005 | 0.005 |
| hydroxybenzoate | | | | | |
| Propyl p- | 0.005 | 0.005 | 0.005 | 0.005 | 0.005 |
| hydroxybenzoate | | | | | |
| Purified water (2) | 46.067 | 45.933 | 45.800 | 46.400 | 46.267 |
| Sum | 100.000 | 100.000 | 100.000 | 100.000 | 100.000 |

TABLE 17
Compositions [weight ratio (w/w %)] of Preparation Examples 81 to 85

| | Preparation | Preparation | Preparation | Preparation | Preparation |
|---|---|---|---|---|---|
| Components | Example 81 | Example 82 | Example 83 | Example 84 | Example 85 |
| Loxoprofen sodium | 3.200 | 3.200 | 3.200 | 3.200 | 3.200 |
| Purified water (1) | 20.000 | 20.000 | 20.000 | 20.000 | 20.000 |
| Carrageenan | 0.133 | 0.133 | 0.133 | 0.133 | 0.013 |
| Locust bean gum | 0.200 | 0.267 | 0.333 | 0.400 | 0.067 |
| Xanthan gum | 0.200 | 0.267 | 0.333 | 0.400 | 0.067 |
| Xylitol | 30.023 | 30.023 | 30.023 | 30.023 | 30.023 |
| Stevioside | 0.100 | 0.100 | 0.100 | 0.100 | 0.100 |
| Methyl p- | 0.005 | 0.005 | 0.005 | 0.005 | 0.005 |
| hydroxybenzoate | | | | | |
| Propyl p- | 0.005 | 0.005 | 0.005 | 0.005 | 0.005 |
| hydroxybenzoate | | | | | |
| Purified water (2) | 46.13333 | 46 | 45.86667 | 45.73333 | 46.52 |
| Sum | 100.000 | 100.000 | 100.000 | 100.000 | 100.000 |

TABLE 18
Compositions [weight ratio (w/w %)] of Preparation Examples 86 to 90

| | Preparation | Preparation | Preparation | Preparation | Preparation |
|---|---|---|---|---|---|
| Components | Example 86 | Example 87 | Example 88 | Example 89 | Example 90 |
| Loxoprofen sodium | 3.200 | 3.200 | 3.200 | 3.200 | 3.200 |
| Purified water (1) | 20.000 | 20.000 | 20.000 | 20.000 | 20.000 |
| Carrageenan | 0.013 | 0.013 | 0.013 | 0.013 | 0.013 |
| Locust bean gum | 0.133 | 0.200 | 0.267 | 0.333 | 0.400 |
| Xanthan gum | 0.133 | 0.200 | 0.267 | 0.333 | 0.400 |
| Xylitol | 30.023 | 30.023 | 30.023 | 30.023 | 30.023 |
| Stevioside | 0.100 | 0.100 | 0.100 | 0.100 | 0.100 |
| Methyl p- | 0.005 | 0.005 | 0.005 | 0.005 | 0.005 |
| hydroxybenzoate | | | | | |
| Propyl p- | 0.005 | 0.005 | 0.005 | 0.005 | 0.005 |
| hydroxybenzoate | | | | | |
| Purified water (2) | 46.387 | 46.253 | 46.120 | 45.987 | 45.853 |
| Sum | 100.000 | 100.000 | 100.000 | 100.000 | 100.000 |

### Test Example 1: Measurement of PAR

Test Method

(i) 1 ml of each formulation sample was filled in a syringe (inner diameter: 12±0.05 mm) having an inlet enlarged by cutting.

(ii) The formulation sample was squeezed and dropped onto an area of 1 cm² on square paper, and then the area of the base (the surface of contact between the formulation and the square paper) was calculated according to (ii) below.

(iii) Calculation of the contact area between the formulation and the square paper.

The top of the formulation dropped on the square paper was photographed. Through the photograph of the formulation, the area of the formulation and the area of the square were calculated as pixel values by use of Image J (V1.46r, manufactured by the National Institutes of Health), and then proportionally expressed using the known square area (25 mm²).

(iv) Based on the area value, the PAR value was calculated using equation 1. The measured PAR value is shown in Table 21 below.

### Test Example 2: Disintegration Test

Each of the formulations of Preparation Examples 1 to 90 was added to a test solution, and the time at which the formulation completely disappeared was considered as disintegration time and recorded in units of seconds.

Test Method

(i) 1 ml of each formulation sample was filled in a syringe (inner diameter: 12±0.05 mm) having an inlet enlarged by cutting.

(ii) Using the formulation, a disintegration test was performed under the following conditions.

Tester: disintegration tester (LABFINE, DIT-200);

Test solution: pH 1.2 solution (solution 1 in the disintegration test method described in the Korean Pharmacopeia); and

Test temperature: 37±0.5° C.

The results of the disintegration test are shown in Table 21 below.

### Test Example 3: Sensory Evaluation

The formulations of Preparation Examples 1 to 90 were subjected to sensory evaluation with respect to bitter taste and swallowing by 10 healthy adult persons. The results of the sensory evaluation were scored according to the criteria shown in Table 19 below, and the average value of the 10 persons is shown in Table 21 below.

TABLE 19
Criteria for Sensory Evaluation

| Score | Bitter taste | Swallowing |
|---|---|---|
| 1 | Severely bitter | The shape is hardly maintained, and swallowing |
| | | is easy. |
| 2 | Bitter | The shape is maintained with some collapse, and |
| | | swallowing is easy. |
| 3 | Slightly bitter | The shape is maintained without collapse, and |
| | | swallowing is easy. |
| 4 | Little bitter | The shape is maintained and slightly hard, but |
| | | there is no rejection when swallowing. |
| 5 | Not bitter | It is hard so that it should be masticated |
| | | before swallowing. |

### Test Example 4: Formulation Discharge Test

10 formulations prepared in each of the Preparation Examples were packed into each stick container and then discharged from the stick container, after which the ease of discharge of the formulation and the amount of formulation residue remaining in the stick container after discharge were evaluated according to the criteria shown in Table 20. The results of the evaluation are shown in Table 21 below.

TABLE 20
Criteria for discharge evaluation

| Score | Discharge | Amount of formulation residue |
|---|---|---|
| 1 | It breaks during discharge | The amount of formulation residue |
| | and is not easily discharged. | in the stick container is large. |
| 2 | It does not break during | There is little formulation residue |
| | discharge and is easily | in the stick container. |
| | discharged. | |
| 3 | — | There is no formulation residue |
| | | in the stick container. |

### Test Example 5: Dissolution Test

In order to examine the dissolution rate of each formulation as a function of the disintegration time, each formulation was added to a test solution, and a dissolution test was performed in the following manner.

Test Method

(i) Each formulation was packed into a stick container (reference mass: 2.5 g).

(ii) Using the formulation, a dissolution test was performed under the following conditions.

Tester: dissolution tester (LABFINE, DST-810);

Test device: paddle (dissolution test method 2 described in the Korean Pharmacopeia);

Paddle rotating speed: 50 rpm;

Test solution: pH 1.2 solution (solution 1 in the dissolution test method described in the Korean Pharmacopeia);

Test time: 30 minutes;

Test temperature: 37±0.5° C.;

Dissolution standards:

Acetaminophen—80% or more for 30 minutes (based on an acetaminophen tablet as described in the Korean Pharmacopeia);

Cetirizine hydrochloride—80(Q) % or more for 30 minutes (based on a Cetirizine hydrochloride tablet as described in the US Pharmacopeia);

Loxoprofen sodium —75% or more for 30 minutes (based on a Loxoprofen sodium tablet as described in the Korean Pharmacopeia).

The results of the dissolution test are shown in Table 21 below.

TABLE 21
Test results of Test Examples

| | | | | | | Discharge test | |
|---|---|---|---|---|---|---|---|
| Preparation | | Disintegration | Dissolution | Sensory test | | | Amount of |
| Examples | PAR(θ) | Time (sec) | Rate (%) | Bitter taste | Swallowing | Discharge | residue |
| Preparation | 48.8 | 22 | 100.6 | 2.0 | 1.2 | 1 | 1.0 |
| Example 1 | | | | | | | |
| Preparation | 53.5 | 36 | 99.9 | 3.6 | 2.7 | 2 | 2.3 |
| Example 2 | | | | | | | |
| Preparation | 66.1 | 69 | 100.1 | 5.0 | 3.0 | 2 | 2.8 |
| Example 3 | | | | | | | |
| Preparation | 68.6 | 115 | 100.6 | 5.0 | 3.4 | 2 | 2.9 |
| Example 4 | | | | | | | |
| Preparation | 70.9 | 152 | 82.1 | 5.0 | 4.6 | 2 | 2.9 |
| Example 5 | | | | | | | |
| Preparation | 70.8 | 156 | 83.4 | 5.0 | 4.5 | 2 | 2.9 |
| Example 6 | | | | | | | |
| Preparation | 41.3 | 19 | 100.4 | 1.7 | 1.0 | 1 | 1.0 |
| Example 7 | | | | | | | |
| Preparation | 56.2 | 39 | 100.6 | 3.8 | 2.9 | 2 | 2.7 |
| Example 8 | | | | | | | |
| Preparation | 67.5 | 63 | 99.8 | 5.0 | 3.3 | 2 | 3.0 |
| Example 9 | | | | | | | |
| Preparation | 68.6 | 107 | 100 | 5.0 | 3.3 | 2 | 3.0 |
| Example 10 | | | | | | | |
| Preparation | 70.9 | 162 | 82.7 | 5.0 | 4.4 | 2 | 3.0 |
| Example 11 | | | | | | | |
| Preparation | 72.1 | 195 | 78.9 | 5.0 | 4.8 | 2 | 3.0 |
| Example 12 | | | | | | | |
| Preparation | 47.7 | 22 | 100.3 | 1.9 | 1.3 | 1 | 1.0 |
| Example 13 | | | | | | | |
| Preparation | 61.5 | 54 | 100.1 | 4.7 | 3.0 | 2 | 3.0 |
| Example 14 | | | | | | | |
| Preparation | 69.4 | 104 | 88.6 | 5.0 | 4.2 | 2 | 3.0 |
| Example 15 | | | | | | | |
| Preparation | 70.2 | 143 | 82.7 | 5.0 | 4.8 | 2 | 3.0 |
| Example 16 | | | | | | | |
| Preparation | 72.8 | 210 | 76.9 | 5.0 | 5.0 | 2 | 3.0 |
| Example 17 | | | | | | | |
| Preparation | 73.2 | 260 | 75.6 | 5.0 | 5.0 | 2 | 3.0 |
| Example 18 | | | | | | | |
| Preparation | 60.1 | 37 | 99.8 | 4.7 | 3.0 | 2 | 3.0 |
| Example 19 | | | | | | | |
| Preparation | 64.3 | 77 | 100 | 4.9 | 3.0 | 2 | 3.0 |
| Example 20 | | | | | | | |
| Preparation | 68.5 | 107 | 100.4 | 5.0 | 3.4 | 2 | 3.0 |
| Example 21 | | | | | | | |
| Preparation | 72.2 | 163 | 77.1 | 5.0 | 4.7 | 2 | 3.0 |
| Example 22 | | | | | | | |
| Preparation | 72.9 | 288 | 76.8 | 5.0 | 4.6 | 2 | 3.0 |
| Example 23 | | | | | | | |
| Preparation | 74.1 | 315 | 72.3 | 5.0 | 4.8 | 2 | 3.0 |
| Example 24 | | | | | | | |
| Preparation | 42.1 | 15 | 100.3 | 1.6 | 1.0 | 1 | 1.0 |
| Example 25 | | | | | | | |
| Preparation | 59.7 | 19 | 100.7 | 4.6 | 3.0 | 2 | 2.9 |
| Example 26 | | | | | | | |
| Preparation | 69.9 | 62 | 89.6 | 5.0 | 3.6 | 2 | 3.0 |
| Example 27 | | | | | | | |
| Preparation | 70.8 | 100 | 81.8 | 5.0 | 3.8 | 2 | 3.0 |
| Example 28 | | | | | | | |
| Preparation | 71.9 | 178 | 79.3 | 5.0 | 4.4 | 2 | 3.0 |
| Example 29 | | | | | | | |
| Preparation | 72.7 | 220 | 76.2 | 5.0 | 4.6 | 2 | 3.0 |
| Example 30 | | | | | | | |
| Preparation | 56.3 | 90 | 98.1 | 2.1 | 1.1 | 1 | 1.0 |
| Example 31 | | | | | | | |
| Preparation | 63.7 | 190 | 95.8 | 3.5 | 2.3 | 2 | 1.9 |
| Example 32 | | | | | | | |
| Preparation | 68.3 | 235 | 85.6 | 4.9 | 2.8 | 2 | 2.8 |
| Example 33 | | | | | | | |
| Preparation | 70.3 | 300 | 77.6 | 5.0 | 3.3 | 2 | 3.0 |
| Example 34 | | | | | | | |
| Preparation | 71.3 | 385 | 74.1 | 5.0 | 4.4 | 2 | 3.0 |
| Example 35 | | | | | | | |
| Preparation | 73.9 | 437 | 70.6 | 5.0 | 4.6 | 2 | 3.0 |
| Example 36 | | | | | | | |
| Preparation | 51.9 | 110 | 97.5 | 2.0 | 1.0 | 1 | 1.0 |
| Example 37 | | | | | | | |
| Preparation | 65.3 | 195 | 93 | 3.7 | 2.6 | 2 | 2.4 |
| Example 38 | | | | | | | |
| Preparation | 66.7 | 266 | 94.2 | 5.0 | 3.2 | 2 | 2.4 |
| Example 39 | | | | | | | |
| Preparation | 72.3 | 340 | 72.1 | 5.0 | 3.3 | 2 | 3.0 |
| Example 40 | | | | | | | |
| Preparation | 74.8 | 395 | 70.1 | 5.0 | 4.2 | 2 | 3.0 |
| Example 41 | | | | | | | |
| Preparation | 75.0 | 455 | 68.3 | 5.0 | 4.5 | 2 | 3.0 |
| Example 42 | | | | | | | |
| Preparation | 55.0 | 115 | 97.5 | 2.5 | 1.4 | 1 | 1.0 |
| Example 43 | | | | | | | |
| Preparation | 64.8 | 208 | 93.9 | 4.7 | 3.2 | 2 | 2.8 |
| Example 44 | | | | | | | |
| Preparation | 66.2 | 270 | 90.1 | 5.0 | 3.7 | 2 | 3.0 |
| Example 45 | | | | | | | |
| Preparation | 72.1 | 344 | 73.5 | 5.0 | 4.5 | 2 | 3.0 |
| Example 46 | | | | | | | |
| Preparation | 73.9 | 394 | 71.3 | 5.0 | 4.8 | 2 | 3.0 |
| Example 47 | | | | | | | |
| Preparation | 75.6 | 505 | 69.7 | 5.0 | 4.9 | 2 | 3.0 |
| Example 48 | | | | | | | |
| Preparation | 65.8 | 123 | 94.8 | 4.2 | 2.5 | 2 | 2.7 |
| Example 49 | | | | | | | |
| Preparation | 67.9 | 224 | 85.7 | 4.4 | 3.0 | 2 | 2.9 |
| Example 50 | | | | | | | |
| Preparation | 72.9 | 289 | 73.1 | 5.0 | 3.4 | 2 | 2.9 |
| Example 51 | | | | | | | |
| Preparation | 73.2 | 347 | 72.5 | 5.0 | 4.2 | 2 | 3.0 |
| Example 52 | | | | | | | |
| Preparation | 74.7 | 430 | 70.8 | 5.0 | 4.7 | 2 | 3.0 |
| Example 53 | | | | | | | |
| Preparation | 74.4 | 507 | 69.9 | 5.0 | 4.9 | 2 | 3.0 |
| Example 54 | | | | | | | |
| Preparation | 56.5 | 102 | 98.9 | 2.0 | 1.4 | 1 | 1.0 |
| Example 55 | | | | | | | |
| Preparation | 64.4 | 190 | 94.2 | 3.8 | 3.0 | 2 | 2.4 |
| Example 56 | | | | | | | |
| Preparation | 68.6 | 240 | 82.4 | 5.0 | 3.0 | 2 | 2.7 |
| Example 57 | | | | | | | |
| Preparation | 70.3 | 312 | 76.1 | 5.0 | 3.4 | 2 | 3.0 |
| Example 58 | | | | | | | |
| Preparation | 72.7 | 387 | 73 | 5.0 | 3.4 | 2 | 3.0 |
| Example 59 | | | | | | | |
| Preparation | 74.7 | 446 | 70.8 | 5.0 | 5.0 | 2 | 3.0 |
| Example 60 | | | | | | | |
| Preparation | 54.4 | 66 | 100.3 | 2.3 | 1.2 | 1 | 1.0 |
| Example 61 | | | | | | | |
| Preparation | 66.4 | 190 | 100.2 | 3.7 | 2.5 | 2 | 2.2 |
| Example 62 | | | | | | | |
| Preparation | 67.3 | 241 | 100.2 | 5.0 | 2.9 | 2 | 2.7 |
| Example 63 | | | | | | | |
| Preparation | 68.9 | 330 | 95.6 | 5.0 | 3.4 | 2 | 3.0 |
| Example 64 | | | | | | | |
| Preparation | 70.9 | 437 | 76.1 | 5.0 | 4.7 | 2 | 3.0 |
| Example 65 | | | | | | | |
| Preparation | 70.7 | 548 | 75.8 | 5.0 | 4.7 | 2 | 2.9 |
| Example 66 | | | | | | | |
| Preparation | 59.8 | 93 | 101.1 | 2.0 | 1.1 | 1 | 1.0 |
| Example 67 | | | | | | | |
| Preparation | 68.1 | 240 | 100.4 | 3.9 | 2.9 | 2 | 2.7 |
| Example 68 | | | | | | | |
| Preparation | 68.3 | 360 | 97.6 | 5.0 | 3.3 | 2 | 3.0 |
| Example 69 | | | | | | | |
| Preparation | 70.1 | 470 | 77.3 | 5.0 | 3.4 | 2 | 3.0 |
| Example 70 | | | | | | | |
| Preparation | 71.6 | 639 | 70.2 | 5.0 | 4.5 | 2 | 3.0 |
| Example 71 | | | | | | | |
| Preparation | 72.6 | 669 | 64.5 | 5.0 | 4.7 | 2 | 3.0 |
| Example 72 | | | | | | | |
| Preparation | 63.0 | 145 | 100.7 | 2.4 | 1.2 | 1 | 1.0 |
| Example 73 | | | | | | | |
| Preparation | 67.2 | 268 | 100.1 | 4.8 | 3.1 | 2 | 3.0 |
| Example 74 | | | | | | | |
| Preparation | 72.0 | 370 | 67.5 | 5.0 | 4.3 | 2 | 3.0 |
| Example 75 | | | | | | | |
| Preparation | 72.1 | 484 | 65.8 | 5.0 | 4.8 | 2 | 3.0 |
| Example 76 | | | | | | | |
| Preparation | 73.6 | 668 | 59.8 | 5.0 | 5.0 | 2 | 3.0 |
| Example 77 | | | | | | | |
| Preparation | 74.7 | 880 | 58.5 | 5.0 | 5.0 | 2 | 3.0 |
| Example 78 | | | | | | | |
| Preparation | 67.4 | 200 | 100.6 | 4.7 | 2.9 | 2 | 3.0 |
| Example 79 | | | | | | | |
| Preparation | 69.8 | 290 | 90.5 | 4.8 | 3.1 | 2 | 3.0 |
| Example 80 | | | | | | | |
| Preparation | 71.2 | 423 | 69.8 | 5.0 | 3.5 | 2 | 3.0 |
| Example 81 | | | | | | | |
| Preparation | 73.2 | 555 | 60.4 | 5.0 | 4.6 | 2 | 3.0 |
| Example 82 | | | | | | | |
| Preparation | 73.6 | 760 | 58.1 | 5.0 | 4.9 | 2 | 3.0 |
| Example 83 | | | | | | | |
| Preparation | 75.0 | 1080 | 57.9 | 5.0 | 5.0 | 2 | 3.0 |
| Example 84 | | | | | | | |
| Preparation | 55.3 | 90 | 100.5 | 2.1 | 1.1 | 1 | 1.0 |
| Example 85 | | | | | | | |
| Preparation | 65.5 | 207 | 100.2 | 4.6 | 3.0 | 2 | 2.9 |
| Example 86 | | | | | | | |
| Preparation | 65.4 | 337 | 100.2 | 5.0 | 3.7 | 2 | 3.0 |
| Example 87 | | | | | | | |
| Preparation | 68.7 | 430 | 91.2 | 5.0 | 3.8 | 2 | 3.0 |
| Example 88 | | | | | | | |
| Preparation | 69.5 | 522 | 83.7 | 5.0 | 4.7 | 2 | 3.0 |
| Example 89 | | | | | | | |
| Preparation | 70.9 | 660 | 77.8 | 5.0 | 4.7 | 2 | 3.0 |
| Example 90 | | | | | | | |

When the PAR value was less than 50, the physical strength of the formulation was excessively low, and thus the formulation easily collapsed in the mouth, indicating that it has little or no effect on masking of the bitter taste of the active ingredient. In addition, in this case, the amount of residue in the stick container after discharge was large, indicating that it is difficult to take a predetermined amount of the active ingredient. On the other hand, when the PAR value was more than 70, the physical strength of the formulation was excessively high, swallowing of the formulation when taking the formulation was not easy, and the formulation did need to be masticated before swallowing, and also the disintegration time was excessively slow, resulting in a low dissolution rate. However, the formulations having a PAR value of 50-70 had suitable physical strength, and thus were easily swallowed, and these formulations maintained their strength at a suitable level in the mouth, and were effective in masking the bitter taste of the active ingredient. In addition, these formulations showed a suitable disintegration time in the disintegration test, and thus satisfied the dissolution standards.

1. A semi-solid formulation for oral administration, comprising an active pharmaceutical ingredient and at least one thickener and having a PAR value of 50-70 as calculated by the following equation 1:
$$PAR ( θ ) = {tan}^{- 1} ( \frac{3 \cdot V}{π {r}^{3}} ) \qquad Equation 1$$
wherein
r is the radius of the base circle of an imaginary cone, and
V: the volume (1 cm³) of the imaginary cone.

2. The semi-solid formulation of claim 1, wherein the thickener is selected from the group consisting of xanthan gum, locust bean gum, guar gum, tragacanth gum, Arabic gum, gellan gum, karaya gum, ghatti gum, tamarind gum, tara gum, acacia gum, agar, chitosan, carrageenan, gelatin, pectin, alginic acid, sodium alginate, propylene glycol alginate, hypromellose, hydroxyethyl cellulose, hydroxypropyl cellulose, methyl cellulose, hydroxyethyl methyl cellulose, sodium carboxymethyl cellulose, calcium carboxymethyl cellulose, ethyl cellulose, polyethylene glycol, polyvinyl alcohol, povidone, polyethylene oxide, and carbopol.

3. The semi-solid formulation of claim 2, wherein the thickener is selected from the group consisting of locust bean gum, guar gum and xanthan gum.

4. The semi-solid formulation of claim 1, further comprising carrageenan.

5. A method for preparing a semi-solid formulation for oral administration according to claim 1, the method comprising the steps of:
(1) dissolving a thickener in a solvent to form a solution;
(2) dispersing or dissolving an active pharmaceutical ingredient in a solvent to form a dispersion or a solution; and
(3) mixing the resultant of step (1) with the resultant of step (2) to form the semi-solid formulation for oral administration.

6. The semi-solid formulation of claim 2, further comprising carrageenan.
